(19) United States
(12) Patent Application Publication
(10) Pub. No.: US 2024/0024324 A1
(43) Pub. Date: Jan. 25, 2024

(54) KINASE INHIBITOR COMBINATIONS FOR CANCER TREATMENT

(71) Applicant: Merck Patent GmbH, Darmstadt (DE)

(72) Inventors: Anderson Clark, Billerica, MA (US); Andreas Machl, Billerica, MA (US); Bayard Huck, Billerica, MA (US); Erik Wilker, Billerica, MA (US); Remigiusz Kaleta, Billerica, MA (US)

(21) Appl. No.: 18/252,897

(22) PCT Filed: Nov. 15, 2021
(86) PCT No.: PCT/EP2021/081612

§ 371 Date: May 15, 2023

Related U.S. Application Data
(63) Continuation of application No. 63/114,144, filed on Nov. 16, 2020.
Application No. 63/114,144 is a continuation of application No. 63/162,649, filed on Mar. 18, 2021.

(51) Int. Cl. A61K 31/517; A61K 31/44; C07K 16/28; A61P 35/00

(52) U.S. Cl. CPC A61K 31/517; A61K 31/44; C07K 16/2863; A61P 35/00; A61K 2039/505

(57) ABSTRACT

A combination includes 4-[(S)-2-Azetidin-1-yl-1-(4-chloro-3-trifluoromethyl-phenyl)-ethylamino]-quinazoline-8-carboxylic acid amide and/or its physiologically acceptable salts and solvates, an inhibitor of MEK kinase, and as an optional third inhibitor, an inhibitor of EGFR. The combination is useful for the treatment of cancer. Another combination includes 4-[(S)-2-Azetidin-1-yl-1-(4-chloro-3-trifloromethyl-phenyl)-ethylamino]-quinazoline-8-carboxylic acid amide and/or its physiologically acceptable salts and solvates, and an inhibitor of EGFR.

The invention relates to combinations of 4-[(S)-2-Azetidin-1-yl-1-(4-chloro-3-trifluoromethyl-phenyl)-ethylamino]-quinazoline-8-carboxylic acid amide (hereinafter referred to as M2698) and/or its physiologically acceptable salts and solvates, and an inhibitor of MEK kinase, and as an optional third inhibitor, with an inhibitor of the receptor tyrosine-protein kinase ERBB-1 also known as EGFR (epidermal growth factor receptor), and the use of such combinations for the treatment of cancer. The invention also relates to combinations of M2698 with an EGFR inhibitor without an inhibitor of MEK kinase.

### BACKGROUND OF THE INVENTION

M2698, processes for its preparation and its use for the treatment of cancer are disclosed in WO 2012/069146 (referred to as Compound A). This compound is a selective, highly potent dual inhibitor of p70S6K and Akt, as demonstrated in a variety of cell-based assays. M2698 was shown to exhibit potent anti-tumor activity against a broad panel of cancer cell lines. Breast cancer cells, glioblastoma multiforme (GBM) cells, endometrial cancer cells and ovarian carcinoma cells have been found to be particularly sensitive to M2698. M2698 crosses the blood-brain barrier in vivo.

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, CA). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)). Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism. Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

### P70S6K Inhibition

Protein kinase 70S6K, the 70 kDa ribosomal protein kinase p70S6K (also known as SK6, p70/p85 S6 kinase, p70/p85 ribosomal S6 kinase and pp70S6K), is a member of the AGC subfamily of protein kinases. p70S6K is a serine-threonine kinase that is a component of the phosphatidylinositol 3 kinase (PI3K)/AKT pathway. p70S6K is downstream of PI3K, and activation occurs through phosphorylation at a number of sites in response to numerous mitogens, hormones and growth factors. p70S6K activity is also under the control of a mTOR-containing complex (TORC1) since rapamycin acts to inhibit p70S6K activity. p70S6K is regulated by PI3K downstream targets Akt and PKCD. Akt directly phosphorylates and inactivates TSC2, thereby activating mTOR. In addition, studies with mutant alleles of p70S6K that are inhibited by Wortmannin but not by rapamycin suggest that the PI3K pathway can exhibit effects on p70S6K independent of the regulation of mTOR activity. The enzyme p70S6K modulates protein synthesis by phosphorylation of the S6 ribosomal protein. S6 phosphorylation correlates with increased translation of mRNAs encoding components of the translational apparatus, including ribosomal proteins and translational elongation factors whose increased expression is essential for cell growth and proliferation. These mRNAs contain an oligopyrimidime tract at their 5′ transcriptional start (termed 5′TOP), which has been shown to be essential for their regulation at the translational level.

In addition to its involvement in translation, p70S6K activation has also been implicated in cell cycle control, neuronal cell differentiation, regulation of cell motility and a cellular response that is important in tumor metastases, the immune response and tissue repair. Antibodies to p70S6K abolish the mitogenic response driven entry of rat fibroblasts into S phase, indicating that p70S6K function is essential for the progression from G1 to S phase in the cell cycle. Furthermore, inhibition of cell cycle proliferation at the G1 to S phase of the cell cycle by rapamycin has been identified as a consequence of inhibition of the production of the hyperphosphorylated, activated form of p70S6K.

A role for p70S6K in tumor cell proliferation and protection of cells from apoptosis is supported based on its participation in growth factor receptor signal transduction, overexpression and activation in tumor tissues. For example, Northern and Western analyses revealed that amplification of the PS6K gene was accompanied by corresponding increases in mRNA and protein expression, respectively (Cancer Res. (1999) 59: 1408-11-Localization of PS6K to Chromosomal Region 17q23 and Determination of Its Amplification in Breast Cancer).

Clinical inhibition of p70S6K activation was observed in renal carcinoma patients treated with CCI-779 (rapamycin ester), an inhibitor of the upstream kinase mTOR. A significant linear association between disease progression and inhibition of p70S6K activity was reported.

In response to energy stress, the tumor suppressor LKB1 activates AMPK which phosphorylates the TSC1/2 complex and enables it to inactivate the mTOR/p70S6K pathway. Mutations in LKB1 cause Peutz-Jeghers syndrome (PJS), where patients with PJS are 15 times more likely to develop cancer than the general population. In addition, 1/3 of lung adenocarcinomas harbor inactivating LKB1 mutations.

Compounds described as suitable for p70S6K inhibition are disclosed in WO 03/064397, WO 04/092154, WO 05/054237, WO 05/056014, WO 05/033086, WO 05/117909, WO 05/039506, WO 06/120573, WO 06/136821, WO 06/071819, WO 06/131835, WO 08/140947, WO 10/093419, WO 12/013282 and WO 12/069146.

It has been shown that M2698 which does not only inhibit p70S6K but also inhibits isoforms 1 and 3 of kinase Akt (upstream of p70S6K in the PI3K pathway) provides more efficient PI3K pathway shutdown (Choo A Y, Yoon S O, Kim S G, Roux P P, Blenis J. Proc. Natl Acad Sci USA. 2008 Nov. 11; 105(45):17414-9.), and allow for capture of any Akt feedback loop activation (Tamburini et al. Blood 2008; 111:379-82).

Inhibition of the PI3K/Akt/mTOR pathway (PAM) is hindered by the subsequent upregulation of the Akt feedback loop (O'Reilly K E et al. Cancer Res. 2006; 66(3):1500-1508). M2698, a selective, dual inhibitor of p70S6K and Akt1/3, blocks signalling downstream of the Akt feedback loop; therefore, M2698 may improve clinical efficacy compared with PAM pathway single-node inhibitors, such as the mTOR inhibitors. M2698 reduces tumor growth and prolongs survival in orthotopically implanted models of human GBM (Machl A et al. Am J Cancer Res. 2016; 6(4):806-818).

The present invention had the objective of finding ways to further advance the pharmaceutical utility for M2698. In this context, combinations of M2698 with an inhibitor of MEK kinase as a dual combination and, in addition, with an inhibitor of EGFR as a triple combination, as well as combinations of M2698 with an inhibitor of EGFR as a dual combination, were studied in vivo.

### MEK Inhibition

The mitogen-activated protein kinase (MAPK) signalling pathway plays critical roles in the regulation of diverse cellular activities, including cell proliferation, survival, differentiation, and motility (Karin L. C. M. Nature. 2001; 410:37-40). Dysregulation of the MAPK pathway occurs in more than one-third of all malignancies. The classical MAPK pathway consists of Ras (a family of related proteins which is expressed in all animal cell lineages and organs), Raf (a family of three serine/threonine-specific protein kinases that are related to retroviral oncogenes), MEK (mitogen-activated protein kinase kinase), and ERK (extracellular signal-regulated kinases), sequentially relaying proliferative signals generated at the cell surface receptors into the nucleus through cytoplasmic signaling. The MEK inhibitor targets the Ras/Raf/MEK/ERK signaling pathway, inhibiting cell proliferation and inducing apoptosis. It hence has potential in clinical use for cancer treatment, especially for those cancers induced by RAS/RAF dysfunction (Leonard J. T. et al., J. Hematol. Oncol. 2016; 9:31. doi: 10.1186/s13045-016-0258-1).

Owing to the widespread activation of this pathway in numerous neoplasms, MEK inhibitors have been in the process of development and study as a type of monotherapy or combination therapy with other targeted and cytotoxic drugs in a variety of clinical situations. More recently, the combination with the use of immune checkpoint inhibitors has emerged as an efficacious treatment for some cancers, expanding the efficacy of this class of agent (Thompson N. et al., Curr. Opin. Pharmacol. 2005; 5:350-356).

Examples of MEK inhibitors under clinical development/and or approved by regulatory authorities include trametinib, cobimetinib, selumetinib, refametinib and pimasertib. Pimasertib is N-(2,3-dihydroxy-propyl)-3-(2-fluoro-4-iodo-phenylamino)-isonicotinamide, and has been described, i.a. in WO2006045514 (Example 115).

Surprisingly, it has been found by the inventors of the present patent application that M2698 acts in a synergistic way when combined with a MEK inhibitor.

### EGFR Inhibition

The epidermal growth factor receptor is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations affecting EGFR expression or activity could result in cancer (Zhang H et al., The Journal of Clinical Investigation. 117 (8): 2051-8). Epidermal growth factor and its receptor was discovered by Stanley Cohen of Vanderbilt University. Cohen shared the 1986 Nobel Prize in Medicine with Rita Levi-Montalcini for their discovery of growth factors. Deficient signalling of the EGFR and other receptor tyrosine kinases in humans is associated with diseases such as Alzheimer's, while over-expression is associated with the development of a wide variety of tumors. Interruption of EGFR signalling, either by blocking EGFR binding sites on the extracellular domain of the receptor or by inhibiting intracellular tyrosine kinase activity, can prevent the growth of EGFR-expressing tumours and improve the patient's condition.

Examples of EGFR inhibitors under clinical development/and or approved by regulatory authorities include gefitinib, erlotinib, afatinib, brigatinib, icotinib, osimertinib and cetuximab (a chimeric (mouse/human) monoclonal antibody, used for the treatment of metastatic colorectal cancer and head and neck cancer).

Surprisingly, it has been found by the inventors of the present patent application that M2698 acts in a synergistic way when combined with a MEK inhibitor and, optionally in addition, an EGFR inhibitor, or when combined with an EGFR inhibitor alone.

### FIGURES

FIG. 1: In vitro analyses in GSC lines. (A) Western blot analysis of protein expression; (B) IC50 of M2698; (C) IC50 of pimasertib; and (D) apoptotic GSC11, GSC7-2, and GSC17 cells after treatment with M2698, pimasertib, or M2698+pimasertib combination. Single agents were tested at their IC50; half the IC50 of each compound was used for the combination.

FIG. 2: (A) Tumor volume and (B) median survival in orthotopic GSC xenograft models, after treatment with vehicle, M2698, pimasertib or M2698+pimasertib combination.

FIG. 3: (A) pS6 expression, (B) pERK expression, and (C) Ki-67 expression in GSC17 and GSC7-2 xenograft models, following treatment with vehicle control, M2698, pimasertib, or M2698+pimasertib combination.

FIG. 4: NSCLC Brain Metastases PDX Models

FIG. 5: Her2+/HR− Breast Cancer PDX Models

FIG. 6: M2698 and Pimasertib combination in 12 PDX models of Cholangiocarcinoma

FIG. 7: M2698, Pimasertib, and Cetuximab in 75 PDX models of CRC (single agent groups)

FIG. 8: M2698, Pimasertib, and Cetuximab in 75 PDX models of CRC (dual combinations)

FIG. 9: M2698, Pimasertib, and Cetuximab in 75 PDX models of CRC (triple combination)

FIG. 10: M2698 and Cetuximab in 38 PDX models of SCCHN (dual combination)

### DETAILED DESCRIPTION OF THE INVENTION

The present invention relates to a method for prophylaxis and/or treatment of cancers, comprising administering to a subject M2698 and/or its physiologically acceptable salts and solvates, a MEK inhibitor and, optionally, an EGFR inhibitor. M2698 and/or its physiologically acceptable salts and solvates, and the other active ingredient(s) can be administered simultaneously or sequentially. When administered simultaneously, M2698 and/or its physiologically acceptable salts and solvates, and the MEK inhibitor may be administered as a compound mixture in one pharmaceutical composition or as separate pharmaceutical compositions.

In a preferred embodiment, the method according to the invention comprises the use of M2698 and/or its physiologically acceptable salts and solvates, the MEK inhibitor and, optionally, the EGFR inhibitor, are administered sequentially.

The present invention relates in particular to a method for prophylaxis and/or treatment of tumors selected from the group consisting of colorectal cancer, breast cancer (in particular of the Her2+/HR− type), cholangiocarcinoma, GBM, SCCHN, and NSCLC (in particular brain metastases of NSCLC).

Moreover, the present invention relates to a pharmaceutical composition, comprising a compound mixture of the active pharmaceutical ingredients (API's) M2698, and physiologically acceptable salts and solvates thereof, and a MEK inhibitor and physiologically acceptable salts and solvates.

Suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methylsulfonates, benzoates or p-toluenesulfonates.

Solvates of M2698 and MEK inhibitors are taken to mean adductions of inert solvent molecules onto M2698 which form owing to their mutual attractive force. Solvates are, for example, hydrates, such as monohydrates or dihydrates, or alcoholates, i.e. addition compounds with alcohols, such as, for example, with methanol or ethanol.

A preferred salt form of M2698 is its free base. Also preferred are its hydrochloride, dihydrochloride, mesylate, succinate or malonate salts.

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence: improved treatment, healing, prevention or elimination of a disease, syndrome, condition, complaint, disorder or prevention of side effects or also reduction in the progress of a disease, condition or disorder. The term “therapeutically effective amount” also encompasses the amounts which are effective for increasing normal physiological function.

The pharmaceutical composition according to the invention comprise mixtures of two API's, for example in the ratio 1:1, 1:2, 1:3, 1:4, 1:5, 1:10, 1:100 or 1:1000. The pharmaceutical composition furthermore comprises at least one solid, liquid and/or semi-liquid excipient or adjuvant. Therefore, the invention also relates to a pharmaceutical composition comprising the said API mixture according to the invention and the said excipients and/or adjuvants.

Furthermore, the present invention relates to the use of the said pharmaceutical composition for the preparation of a medicament for the treatment of cancer.

The invention also relates to a set (kit) consisting of separate packs of
(a) a pharmaceutical composition comprising an effective amount of M2698,
(b) a pharmaceutical composition comprising an effective amount of a MEK inhibitor and, optionally,
(c) a pharmaceutical composition comprising an effective amount of an EGFR inhibitor.

The invention also relates to a set (kit) consisting of separate packs of
(a) a pharmaceutical composition comprising an effective amount of M2698, and
(b) a pharmaceutical composition comprising an effective amount of an EGFR inhibitor.

The set comprises suitable containers, such as boxes, individual bottles, bags or ampoules. The set may, for example, comprise separate ampoules, each containing a pharmaceutical composition comprising an effective amount of M2698 and/or pharmaceutically usable salts thereof, a pharmaceutical composition comprising an effective amount of the MEK inhibitor and/or pharmaceutically usable salts thereof and, optionally, a pharmaceutical composition comprising an effective amount of the EGFR inhibitor in dissolved or lyophilised form.

A particularly preferred, brain penetrant MEK inhibitor to be combined with M2698 is pimasertib. A particularly preferred EGFR inhibitor to be combined with M2698, with or without the MEK inhibitor is cetuximab. A particularly preferred triple combination is M2698+pimasertib+cetuximab.

The compounds and compound mixtures according to the invention can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such medicaments can be prepared using all processes known in the pharmaceutical art by, for example, combining the active ingredient with the excipient(s) or adjuvant(s).

Compounds and compound mixtures adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the compound or compound mixtures can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the compound or compound mixtures after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds and compound mixtures according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compounds and compound mixtures in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil, or natural sweeteners or saccharin or other artificial sweeteners, and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds and compound mixtures according to the invention and salts and solvates thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds and compound mixtures according to the invention can also be delivered using monoclonal antibodies as individual carriers to which the compound molecules are coupled. The compounds and compound mixtures can also be coupled to soluble polymers as targeted medicament carriers. Such polymers may encompass polyvinylpyrrolidone, pyran copolymer, polyhydroxypropylmethacryl-amidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Compounds and compound mixtures adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6):318, 1986.

Compounds and compound mixtures adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the compounds or compound mixtures can be employed either with a paraffinic or a water-miscible cream base. Alternatively, the compounds or compound mixtures can be formulated to give a cream with an oil-in-water cream base or a water-in-oil base.

Compounds and compound mixtures adapted for topical application to the eye include eye drops, in which the active ingredient is dissolved or suspended in a suitable carrier, in particular an aqueous solvent.

Compounds and compound mixtures adapted for topical application in the mouth encompass lozenges, pastilles and mouthwashes.

Compounds and compound mixtures adapted for rectal administration can be administered in the form of suppositories or enemas.

Compounds and compound mixtures adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Compounds and compound mixtures adapted for administration by inhalation encompass finely particulate dusts or mists, which can be generated by various types of pressurised dispensers with aerosols, nebulisers or insufflators.

Compounds and compound mixtures adapted for vaginal administration can be administered as pessaries, tampons, creams, gels, pastes, foams or spray formulations.

Compounds and compound mixtures adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the medicaments according to the invention may also comprise other agents usual in the art with respect to the particular type of pharmaceutical formulation; thus, for example, compounds or compound mixtures which are suitable for oral administration may comprise flavours.

A therapeutically effective amount of a compound or compound mixture of the present invention depends on a number of factors, including, for example, the age and weight of the recipient, the precise condition that requires treatment, and its severity, the nature of the formulation and the method of administration, and is ultimately determined by the treating doctor or vet. However, an effective amount of an API for the treatment of the diseases according to the invention is generally in the range from 0.1 to 100 mg/kg of body weight of the recipient (mammal) per day and particularly typically in the range from 1 to 10 mg/kg of body weight per day.

Thus, the actual amount per day for an adult mammal weighing 70 kg is usually between 70 and 700 mg, where this amount can be administered as an individual dose per day or more usually in a series of part-doses (such as, for example, two, three, four, five or six) per day, so that the total daily dose is the same. An effective amount of a salt or solvate or of a physiologically functional derivative thereof can be determined as a fraction of the effective amount of the compounds and compound mixtures according to the invention per se.

The pharmaceutical preparations according to the invention can be employed as medicaments in human and veterinary medicine. Suitable excipients are organic or inorganic substances which are suitable for enteral (for example oral), parenteral or topical administration and do not react with the novel compounds, for example water, vegetable oils, benzyl alcohols, polyethylene glycols, gelatine, carbohydrates, such as lactose or starch, magnesium stearate, talc or Vaseline. Suitable for enteral administration are, in particular, tablets, coated tablets, capsules, syrups, juices, drops or suppositories, suitable for parenteral administration are solutions, preferably oil-based or aqueous solutions, furthermore suspensions, emulsions or implants, and suitable for topical application are ointments, creams or powders. The compounds and compound mixtures may also be lyophilised and the resultant lyophilisates used, for example, for the preparation of injection preparations.

The preparations indicated may be sterilised and/or comprise adjuvants, such as lubricants, preservatives, stabilisers and/or wetting agents, emulsifiers, salts for modifying the osmotic pressure, buffer substances, dyes, flavours and/or aroma substances. They can, if desired, also comprise one or more further active ingredients, for example one or more vitamins.

The present invention also relates to a method for prophylaxis and/or treatment of cancers, especially squamous cell carcinoma of the head and neck (SCCHN), comprising administering to a subject M2698 and/or its physiologically acceptable salts and solvates, and an EGFR inhibitor, especially cetuximab, as well as corresponding compound mixtures, pharmaceutical compositions, formulations and ways of administration, as described hereinabove.

In certain embodiments, the invention relates to:
1. Compound mixture, comprising 4-[(S)-2-Azetidin-1-yl-1-(4-chloro-3-trifluoromethyl-phenyl)-ethylamino]-quinazoline-8-carboxylic acid amide, or physiologically acceptable salts thereof, and an inhibitor of MEK, or physiologically acceptable salts thereof.
2. Compound mixture, as described above in this enumeration of embodiments, wherein the MEK inhibitor is pimasertib.
3. Compound mixture, as described above in this enumeration of embodiments, further comprising an EGFR inhibitor.
4. Pharmaceutical composition, comprising a compound mixture as described above in this enumeration and, optionally, excipients and/or adjuvants.
5. Set (kit) comprising separate packs of
   (a) an effective amount of 4-[(S)-2-Azetidin-1-yl-1-(4-chloro-3-trifluoromethyl-phenyl)-ethylamino]-quinazoline-8-carboxylic acid amide or physiologically acceptable salts thereof and
   (b) an effective amount of a MEK inhibitor, or physiologically acceptable salts thereof.
6. Set (kit), as described above in this enumeration of embodiments, further comprising a separate pack of
   (c) an effective amount of an EGFR inhibitor or physiologically acceptable salts thereof.
7. Set (kit), as described above in this enumeration of embodiments, wherein the EGFR inhibitor is cetuximab.
8. Method for prophylaxis or treatment of cancer comprising administering to a subject 4-[(S)-2-Azetidin-1-yl-1-(4-chloro-3-trifluoromethyl-phenyl)-ethylamino]-quinazoline-8-carboxylic acid amide, or physiologically acceptable salts thereof, and a MEK inhibitor, or physiologically acceptable salts thereof.
9. Method, as described above in this enumeration of embodiments wherein the MEK inhibitor is pimasertib.
10. Method, as described above in this enumeration of embodiments, further comprising administering to a subject an EGFR inhibitor, or physiologically acceptable salts thereof.
11. Method, as described above in this enumeration of embodiments, wherein the EGFR inhibitor is cetuximab.
12. Method, as described above in this enumeration of embodiments, wherein 4-[(S)-2-Azetidin-1-yl-1-(4-chloro-3-trifluoromethyl-phenyl)-ethylamino]-quinazoline-8-carboxylic acid amide, the MEK inhibitor and, optionally, the EGFR inhibitor, are administered simultaneously.
13. Method, as described above in this enumeration of embodiments, wherein 4-[(S)-2-Azetidin-1-yl-1-(4-chloro-3-trifluoromethyl-phenyl)-ethylamino]-quinazoline-8-carboxylic acid amide, the MEK inhibitor and, optionally, the EGFR inhibitor, are administered sequentially.
14. Compound mixture, comprising 4-[(S)-2-Azetidin-1-yl-1-(4-chloro-3-trifluoromethyl-phenyl)-ethylamino]-quinazoline-8-carboxylic acid amide, or physiologically acceptable salts thereof, and an inhibitor of EGFR, preferably cetuximab.
15. Set (kit) comprising separate packs of
   (a) an effective amount of 4-[(S)-2-Azetidin-1-yl-1-(4-chloro-3-trifluoromethyl-phenyl)-ethylamino]-quinazoline-8-carboxylic acid amide or physiologically acceptable salts thereof and
   (b) an effective amount of a EGFR inhibitor, preferably cetuximab.
16. Method for prophylaxis or treatment of cancer comprising administering to a subject 4-[(S)-2-Azetidin-1-yl-1-(4-chloro-3-trifluoromethyl-phenyl)-ethylamino]-quinazoline-8-carboxylic acid amide, or physiologically acceptable salts thereof, and an EGFR inhibitor, preferably cetuximab, wherein the two agents are either administered simultaneously or sequentially.
17. In preferred embodiments the cancer is colorectal cancer, breast cancer, cholangiocarcinoma, glioblastoma multiforme, squamous cell carcinoma of the head and neck, and non-small lung cancer.

### Examples

### Example 1: Combination of M2698 and Pimasertib in Patient Derived Xenograft Models (PDX) of Glioblastoma (GBM)

### GSC Lines: Sensitivity to M2698 and Pimasertib In Vitro

Although the effects of M2698 in some GBM models were significant, the responses were relatively modest. As both the PAM and MAPK pathways are involved in GBM, M2698 was combined with brain-penetrant MEK inhibitor pimasertib19 GSC (glioblastoma stem cell) cells/models, in vivo and in vitro.

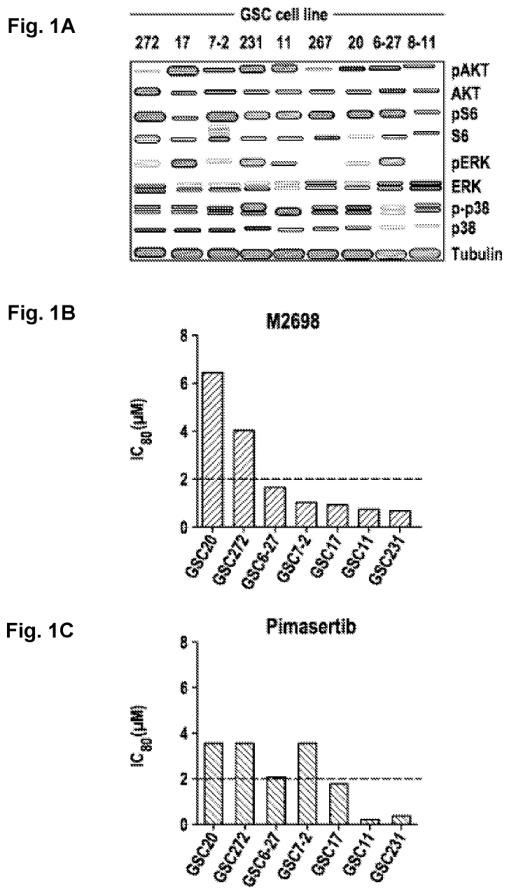

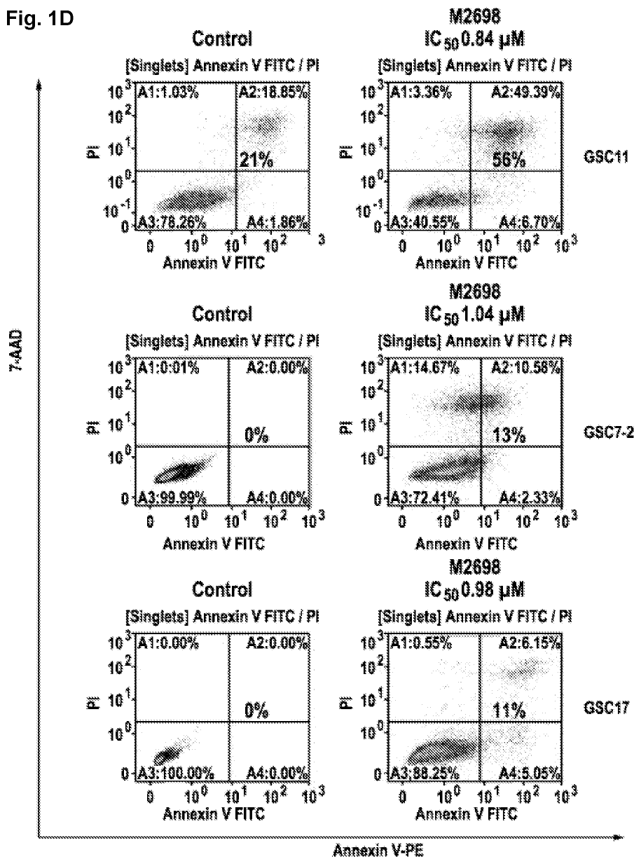

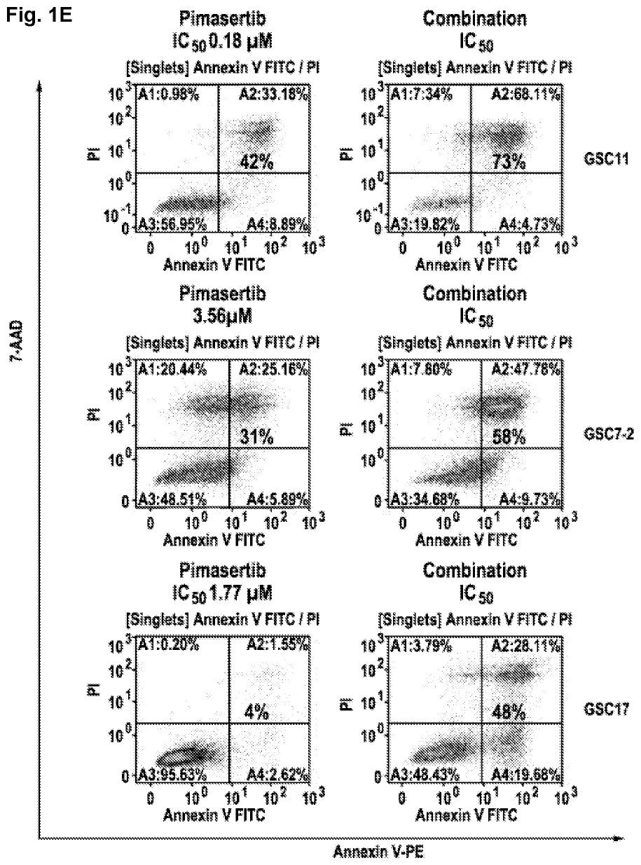

In vitro IC50 data were used to select GSC lines for in vivo studies; IC50 values <2 μM were considered sensitive and >2 μM were considered resistant. All GSC lines expressed Akt. Relatively high pAkt expression in GSC17, GSC7-2, GSC231, GSC11, GSC20, GSC6-27, and GSC8-11 lines compared with GSC272 and GSC267 (FIG. 1A). The cell lines with relatively greater pAkt expression were sensitive to M2698 except for GSC20, suggesting that constitutive Akt activation sensitized cells to M2698 (FIG. 1B). Fewer cell lines (GSC17, GSC11, and GSC231) were sensitive to pimasertib (IC50<2 μM), with GSC20, GSC272, GSC6-27, and GSC7-2 being resistant (FIG. 1C).

Induction of apoptosis by M2698, pimasertib, and the combination was measured in three of five GSC lines that were sensitive to M2698 in vitro. Of those, GSC11 was the most sensitive to pimasertib, GSC17 was moderately sensitive, and GSC7-2 was not sensitive to pimasertib. Both M2698 and pimasertib administered as monotherapy in GSC11, GSC17, and GSC7-2 lines induced apoptosis, although there was no apparent differences in the apoptotic percentages at the IC50 (56%, 11%, 13%, respectively) vs 2×IC50 (62%, 12%, 17%, respectively) for each compound. M2698+pimasertib had either additive (GSC11 and GSC7-2 lines), or apparent synergy (GSC17 line) in induction of apoptosis, compared with either single agent alone (FIG. 1D).

In Vivo Efficacy and PD effects of M2698 and Pimasertib in Orthotopic GSC Xenograft Models

The in vivo effects of M2698 and pimasertib, alone and in combination, were assessed in the GSC models after orthotopic implantation into mice. Treatment duration and time of euthanasia post-implantation was 5-10 weeks. Endpoints were expression of PD markers pS6 and pERK, proliferation marker Ki67, apoptosis as measured by TUNEL, tumor volume and survival.

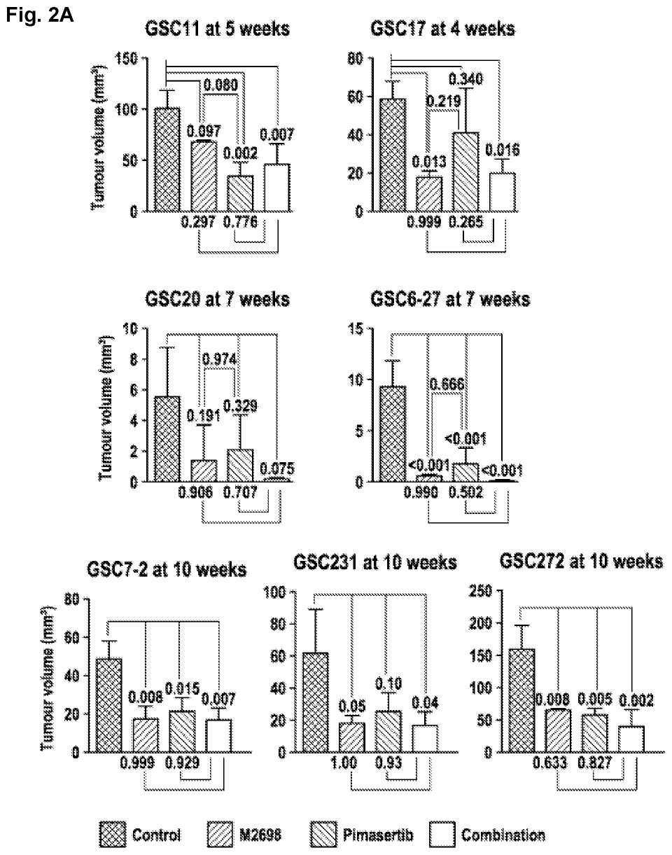

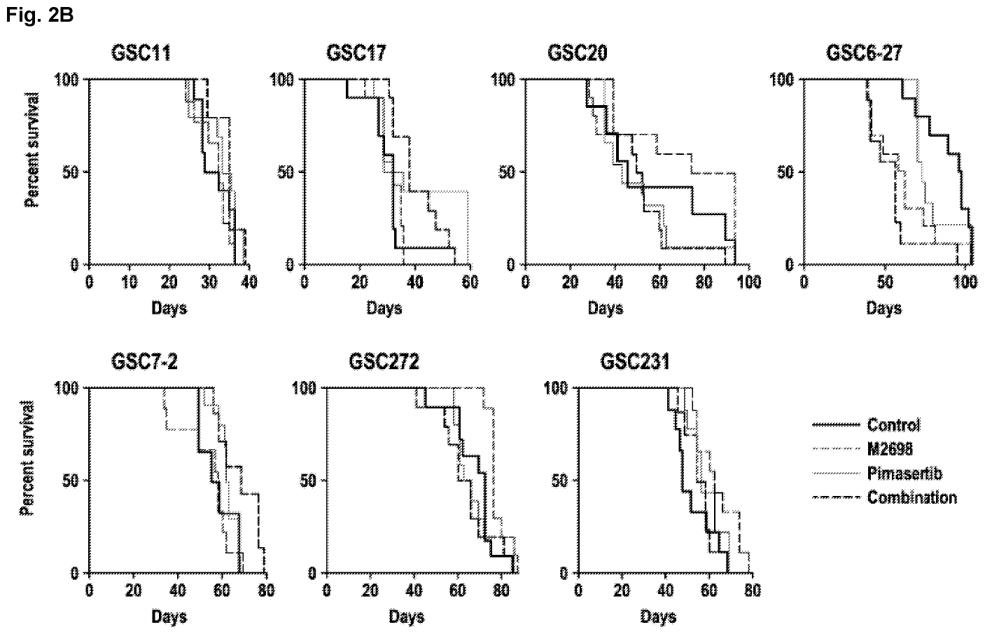

All but one (GSC20) of the seven GSC models were sensitive to M2698 monotherapy as measured by tumor volume; M2698 either significantly inhibited tumor growth (GSC17, GSC6-27, GSC7-2, GSC272, GSC231; all P<0.05; FIG. 2A) or had a trend to do so (GSC11, P=0.10). Of the six M2698-sensitive models, five responded to pimasertib monotherapy, either with significant tumor growth inhibition (GSC6-27, GSC7-2, GSC272; P<0.05) or with a trend towards a significant response (GSC231 P=0.10). Tumor growth of GSC17 was not affected by pimasertib alone (P=0.34 compared to vehicle). M2698+pimasertib significantly inhibited tumor growth in all GSC models compared to vehicle, even in GSC20 (all P<0.05; FIG. 2A) which had not responded to the monotherapy treatments with regard to tumor growth (P>0.05).

Median survival of the GSC models was not entirely reflective of the tumor volume data. (FIG. 1B). M2698 (GSC272 and GSC231) and the combination (GSC17 and GSC7-2) each prolonged survival in only two models compared to vehicle (P<0.05) and pimasertib had no significant effects on survival in any of the models versus controls (P>0.05). In GSC6-27, the vehicle-treated mice had significantly longer median survival than mice from the treatment groups (P<0.05) for unknown reasons. In contrast, both monotherapies significantly inhibited tumor growth in some models and the combination inhibited tumor growth in every model.

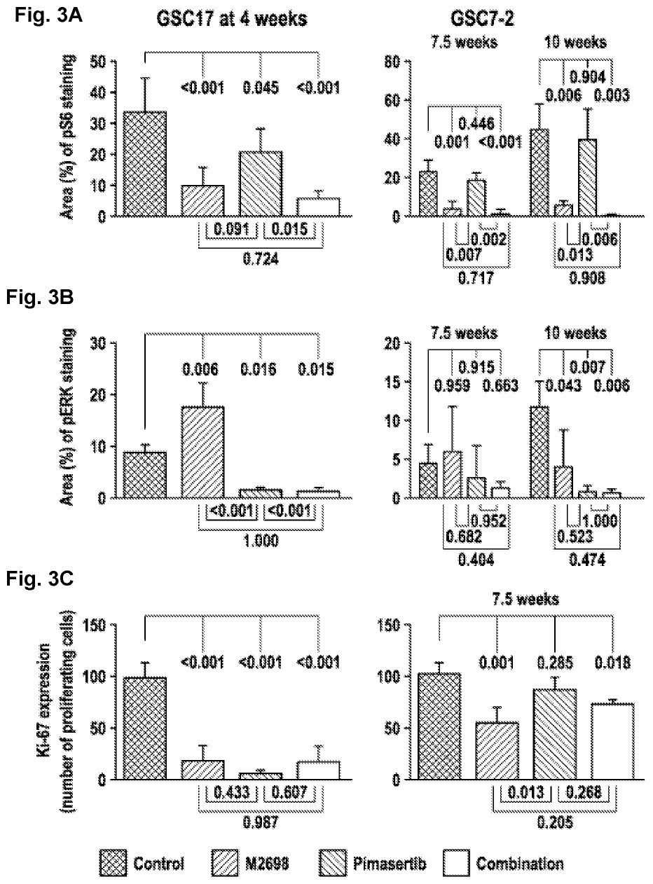

The significant effects on tumor volume indicate that the compounds entered the brain and affected the orthotopically growing tumors. Additional evidence of BBB penetration included the effects on PD markers of the targeted signaling pathways in tumor cells. M2698 significantly reduced pS6 in the orthotopic tumors of all GSC models (P<0.05; FIG. 3A) except for GSC20. Similarly, significant effects of pimasertib treatment on pERK protein were not seen in the GSC20 tumor (P=0.18) whereas pimasertib significantly reduced pERK in the other GSC tumors, (P<0.05; FIG. 3B) including GSC17 which did not experience a reduced tumor volume following pimasertib treatment. The M2698+pimasertib combination reduced pS6 and pERK in all models (all P<0.05) except for GSC20. The relatively high variation in tumor growth, pS6 and pERK in the vehicle-treated mice of the GSC20 model might have masked statistical significance of biologically relevant treatment effects on these endpoints. Conversely, the lack of significant response was predicted by insensitivity of GSC20 to both compounds in vitro (FIG. 1B and FIG. 1C).

In addition to effects of pimasertib on pERK and M2698 on pS6 in the orthotopic brain models, each M2698 also was able to affect the opposite PD marker. Pimasertib significantly reduced pS6 in the GSC272 and GSC231 models. M2698 reduced pERK in all GSC models except GSC20 (P<0.05; FIG. 3B).

Three of the GSC tumors were analyzed at more than one time point with regard to PD markers. Although statistical analysis for responses across time were not performed, trends in different endpoints were evaluated. The percentage of pS6-positive cells increased over time in the GSC7-2 and GSC272 tumors but not GSC231 tumors, while pERK increased only in the GSC7-2 tumors over time (FIG. 3). The cross-pathway reduction of pS6 by pimasertib in the GSC272 and GSC231 models diminished from 7.5-10 weeks and the activity of the compounds on pERK expression, alone or in combination, reduced from 7.5-10 weeks in the GSC231 model. Whether these trends are related to development of resistance would require additional study, but they are reflective of the heterogeneity of the models. Ki67 was measured by immunohistochemistry (FIG. 3C) in sections of tumor from GSC17 and GSC7-2, the two models in which the M2698+pimasertib combination significantly prolonged survival. In the GSC17 model, all three treatments significantly reduced proliferation compared to vehicle, as did M2698 monotherapy and its combination with pimasertib in the GSC7-2 model (all P<0.05).

Neither M2698, pimasertib, nor combination treatment significantly affected apoptosis in GSC7-2 xenografts vs control at 7.5 weeks. After 10 weeks, however, similar to in vitro data, the M2698+pimasertib combination significantly increased in the number of apoptotic cells/field (172.33±60.80) compared with control (8.50±1.80; P=0.002), M2698 alone (52.17±25.20; P=0.01), and pimasertib alone (65.00±60.80; P=0.02).

### Example 2: Combination of M2698 and Pimasertib in Patient Derived Xenograft Models (PDX) of Non-Small Lung Cancer (NSCLC) Brain Metastases

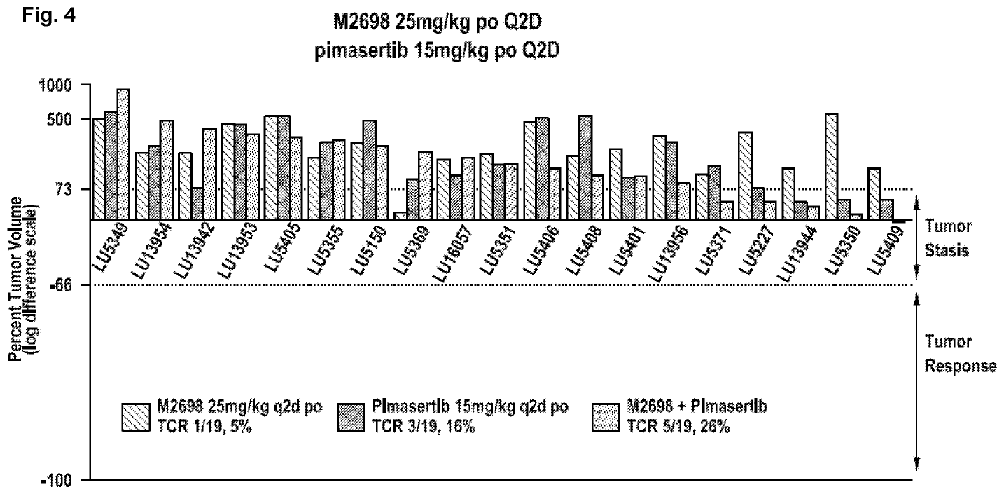

As can be seen in FIG. 4, the combination of M2698 with pimasertib, unexpectedly, shows a tumor control rate (TCR) of 26%, while the single agents show 5% (M2698) and 16% (pimasertib).

### Example 3: Combination of M2698 and Pimasertib in Patient Derived Xenograft Models (PDX) of Her2+/HR− Breast Cancer

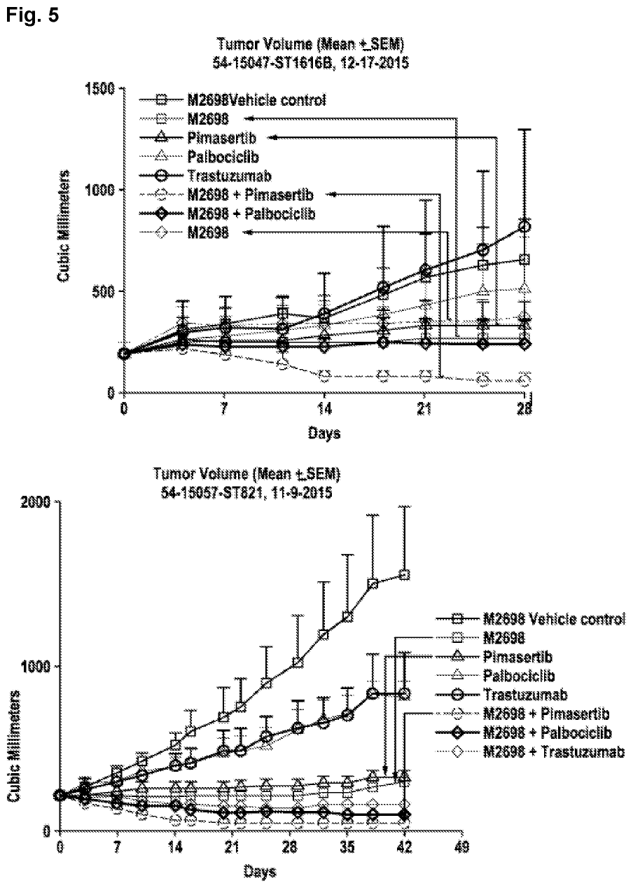

As can be seen in FIG. 5, the combination of M2698 with pimasertib, unexpectedly, decreases the tumor volume to almost zero over the duration of the experiment, while the single agents merely achieve stasis of the tumor volume.

### Example 4: Combination of M2698 and Pimasertib in Patient Derived Xenograft Models (PDX) of Cholangiocarcinoma

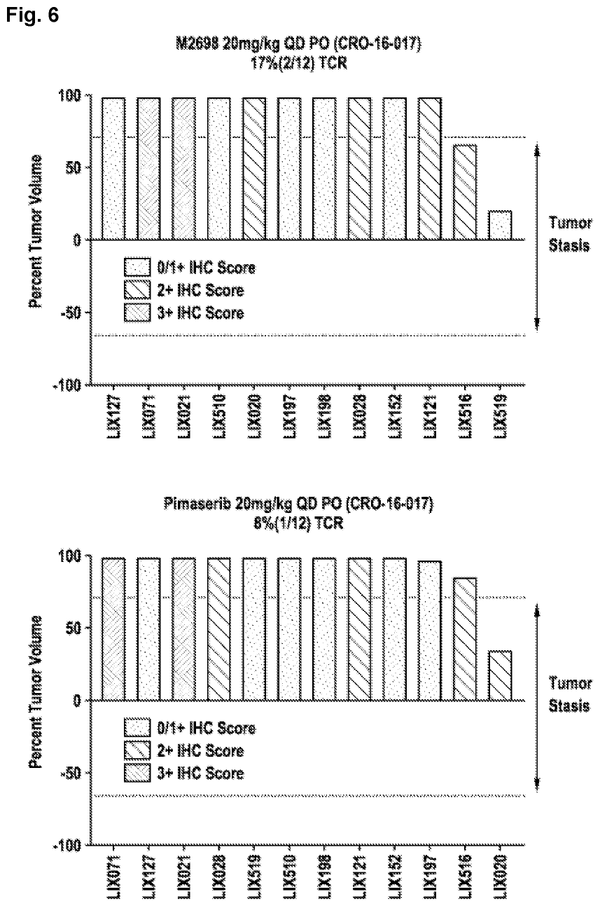

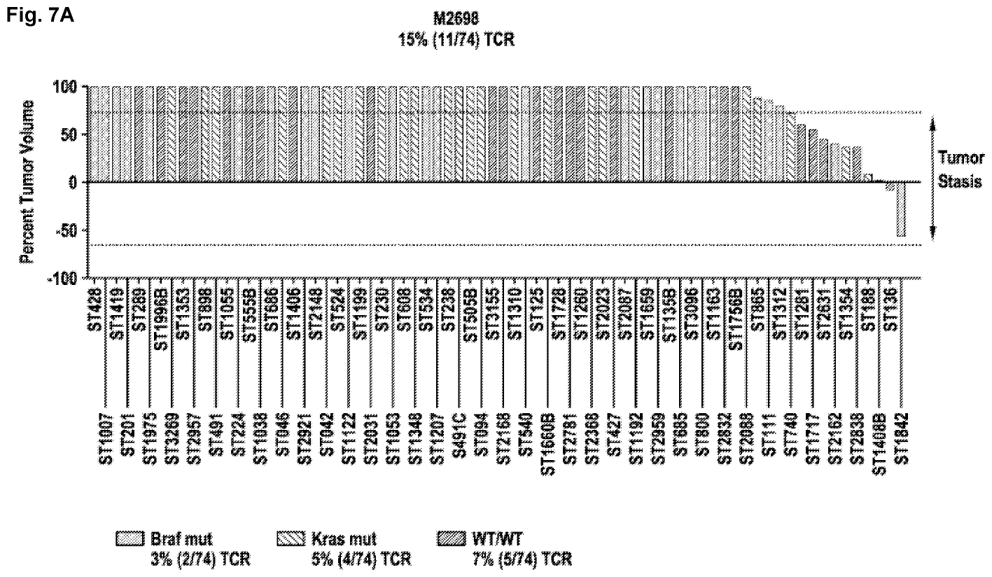

The objective of this experiment was to evaluate the antitumor activity of M2698 alone and in combination with Pimasertib in a panel of 13 Patient-Derived Xenograft models representing human cholangiocarcinoma cancer derived from Chinese patients in immune-deficient mice. The Her2 status of the models was obtained via immunohistochemistry (IHC), and is displayed in FIG. 6.

### Study Design

| | | Dosage | Volume | | | n |
|---|---|---|---|---|---|---|
| | Group | (mg/kg) | (ml/kg) | Schedule | Route | mice |
| 1 | Vehicle | — | 10 | QD | PO | 3 |
| 2 | M2698 | 20 | 10 | QD | PO | 3 |
| 3 | Pimasertib | 20 | 10 | QD | PO | 3 |
| 4 | M2698 + Pimasertib | 20 + 20 | 10 | QD | PO | 3 |

Vehicle: 0.5% Methocel/0.25% Tween20 in Milli-Q Water. Tumors from three untreated mice were collected as satellite samples when reached a tumor volume between 300-500 mm3.

As can be derived from FIG. 6, surprisingly, tumor stasis is achieved with M2698 and pimasertib in 10 of 12 tumor samples (83%), while the single agents show 17% (2/12, M2698) and 8% (1/12, pimasertib). Tumor growth response, or even regression was not observed in this experiment in either treatment.

### Example 5: Combinations of M2698, Pimasertib and Cetuximab in 75 Patient Derived Xenograft Models (PDX) of Colorectal Cancer

### Study Design

| | | Dose | | Dosing | N per |
|---|---|---|---|---|---|
| Group | Treatment | (mg/kg) | Route | schedule | group |
| 1 | Vehicle | | po | qd | 5 |
| 2 | M2698 | 25 | po | qd | 3 |
| 3 | Pimasertib | 20 | po | qd | 3 |
| 4 | Cetuximab | 20 | ip | q2w | 3 |
| 5 | M2698 + Pimasertib | 25 + 20 | po + po | qd + qd | 3 |
| 6 | M2698 + Cetuximab | 25 + 20 | po + ip | qd + q2w | 3 |
| 7 | Pimasertib + | 20 + 20 | po + ip | qd + q2w | 3 |
| | Cetuximab | | | | |
| 8 | M2698 + Pimasertib + | 25 + | po + | qd + | 3 |
| | Cetuximab | 20 + 20 | po ++ ip | qd + q2w | |

Of the 75 models, 25 were wild type, 25 KRas mutant, and 25 BRaf mutant.

### Results

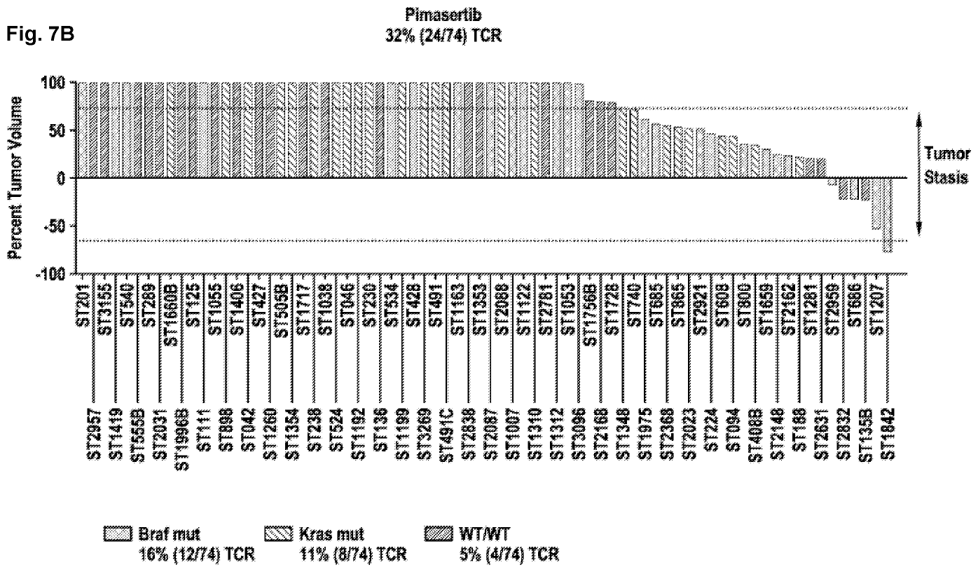

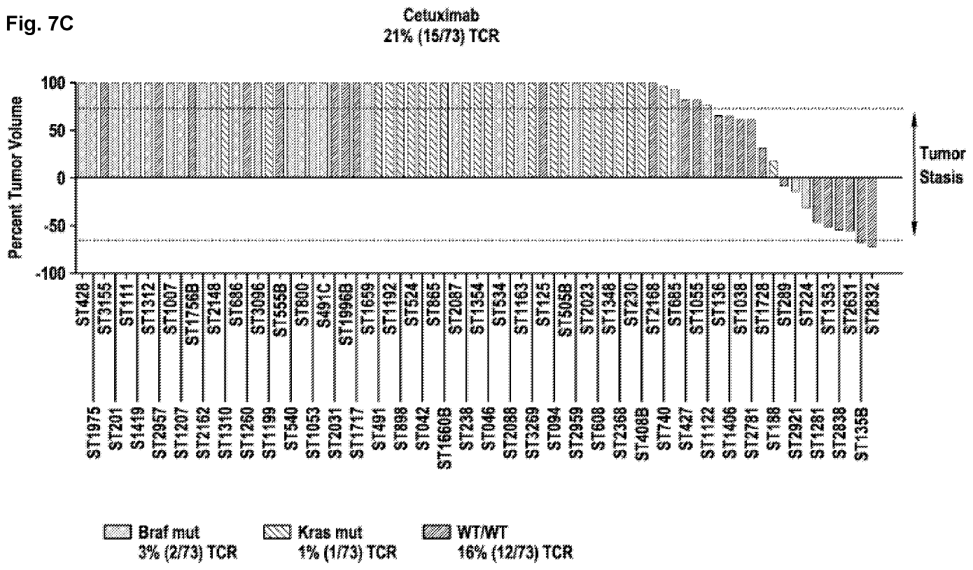

As FIG. 7 shows, for the single agents the tumor control rates (TCR) were 15% (11/74) for M2698, 32% (24/74) for Pimasertib and 21% (15/73) for Cetuximab.

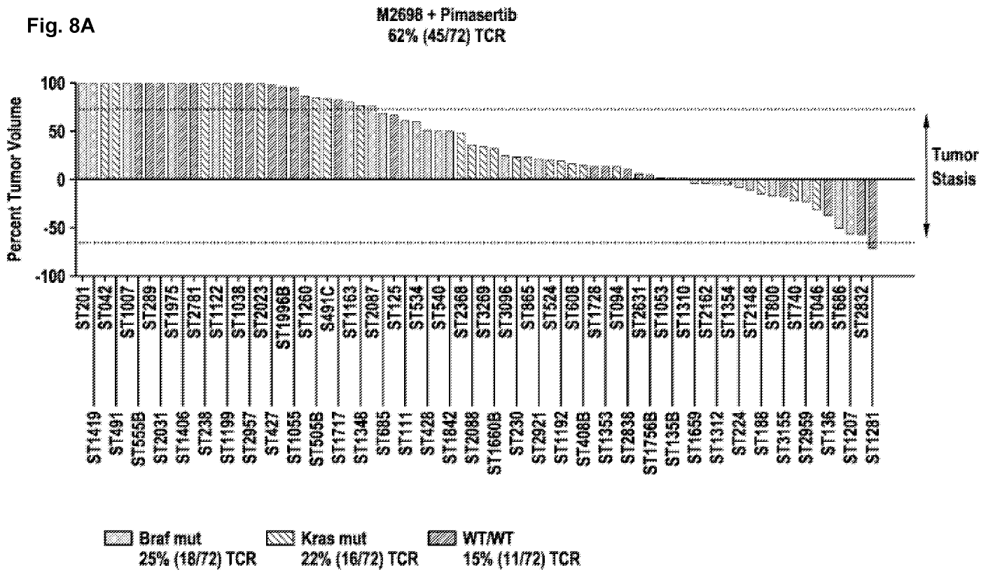

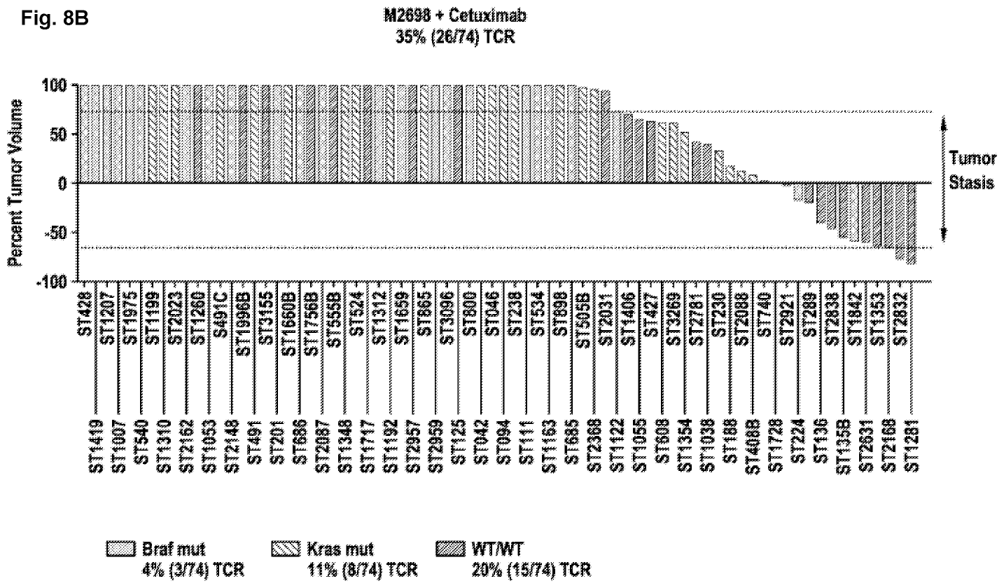

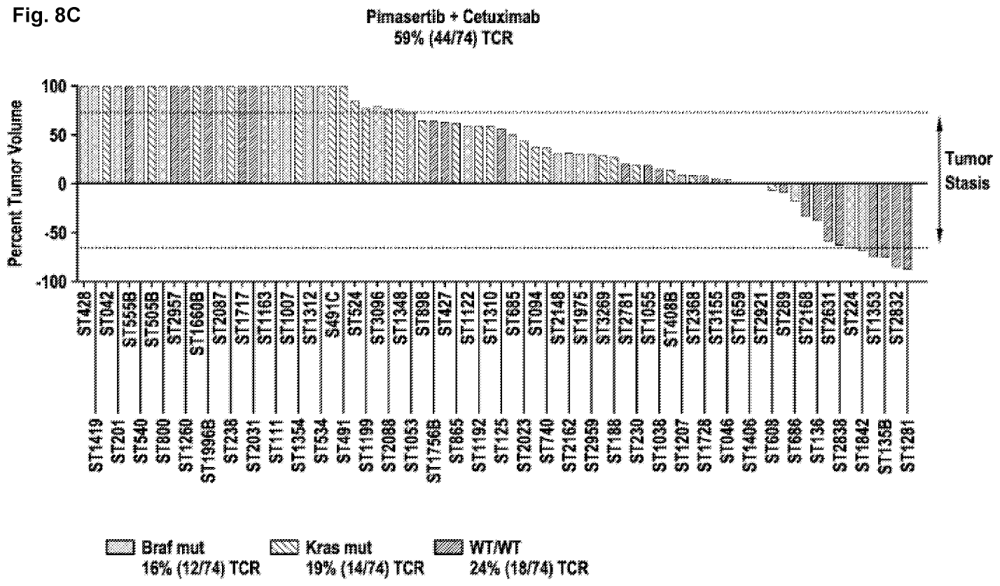

As FIG. 8 shows, for the dual combinations, the TCR's were 59% (44/74) for Pimasertib+Cetuximab, and 35% (26/74) for M2698+Cetuximab and, unexpectedly, 63% (45/72) for M2698+Pimasertib.

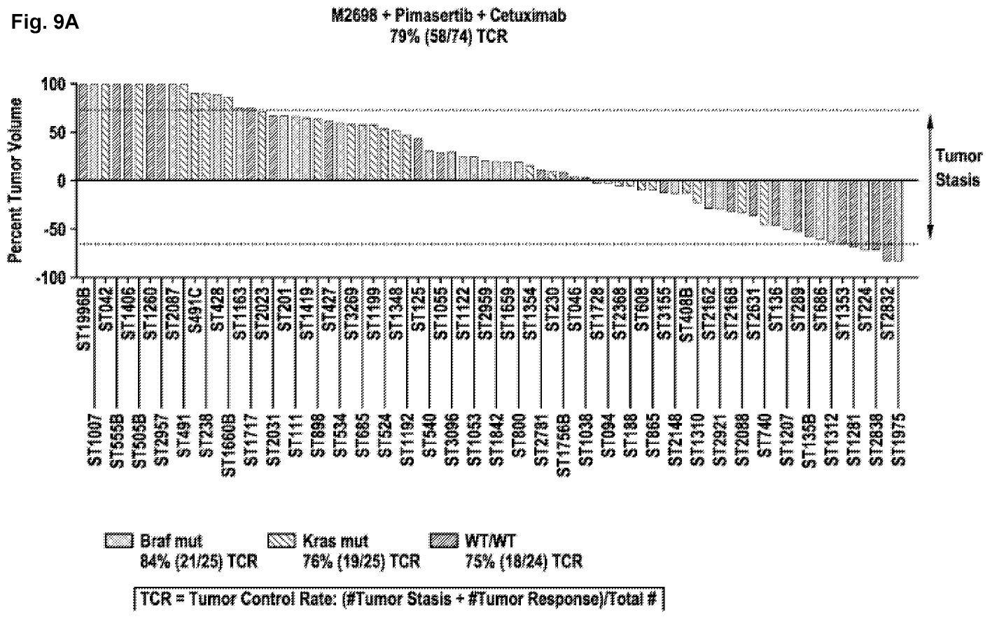

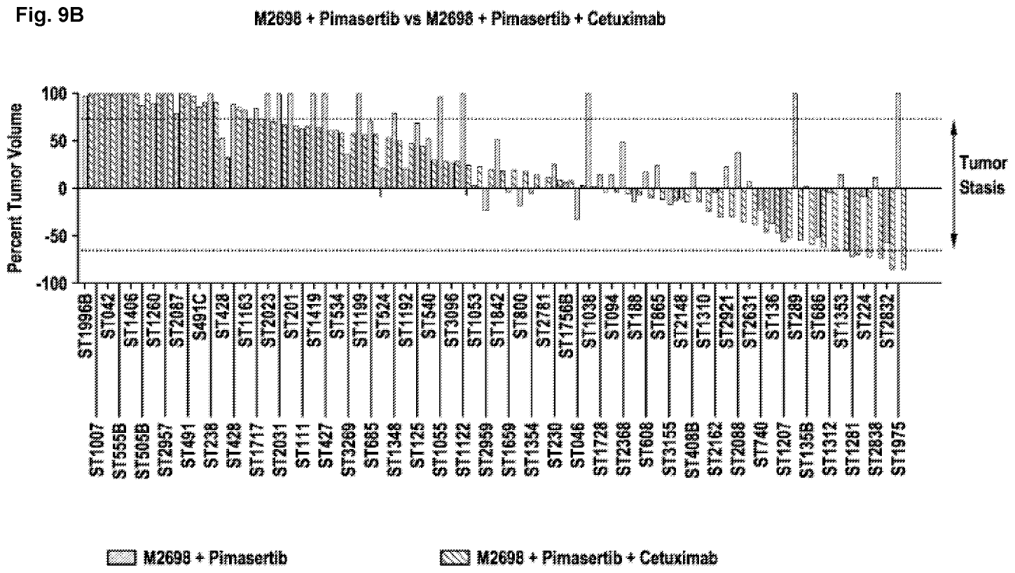

As FIG. 9 shows, for the triple combination, surprisingly the TCR is 78% (58/74), which is again significantly higher than the TCR's of the dual combinations.

### Example 6: Combinations of M2698 and Cetuximab in 38 Patient Derived Xenograft Models (PDX) of SCCHN

46 SCCHN PDX models were run in a 1+1 scheme (one mouse per treatment group). 8 models were cancelled because the models failed to grow. With the adjustment to the cancellation, 38 models were completed. M2698 was administered with standard-of-care (SoC) agents cisplatin and cetuximab in all monotherapies and combinations in a “1+1” screen, i.e. one mouse per treatment per model. After implantation, treatments began when average tumor volume reached ˜200 mm³ for each model. Tumor volumes were measured for each model until the control tumor reached ˜1200 mm³ at which time all animals for that model were euthanized.

### Study Design

| | | | | Dose | | |
|---|---|---|---|---|---|---|
| | | | Dose | Volume | | |
| | | | (mg/kg/ | (mL/kg/ | | |
| Group | n | Agent | dose) | dose) | ROA | Schedule |
| 1 | 1 | Vehicle | — | 10 | PO | QDx28 |
| 2 | 1 | M2698 | 25 | 10 | PO | QDx28 |
| 3 | 1 | Cetuximab | 30 | 10 | IP | 2wklyx4 |
| 4 | 1 | M2698 | 25 | 10 | PO | QDx28 |
| | | Cetuximab | 30 | 10 | IP | 2wklyx4 |

### Results

| | | | M2698 + |
|---|---|---|---|
| Tumor growth Response | M2698 | Cetuximab | Cetuximab |
| Tumor Progression | 31 | 21 | 16 |
| Tumor Stasis | 7 | 14 | 17 |
| Partial response | 0 | 3 | 5 |
| Models with an Overall | 7/38 | 17/38 | 22/38 |
| Response (“RECIST”) | | | |
| Tumor Control Rate (TCR) | 18% | 44% | 58% |

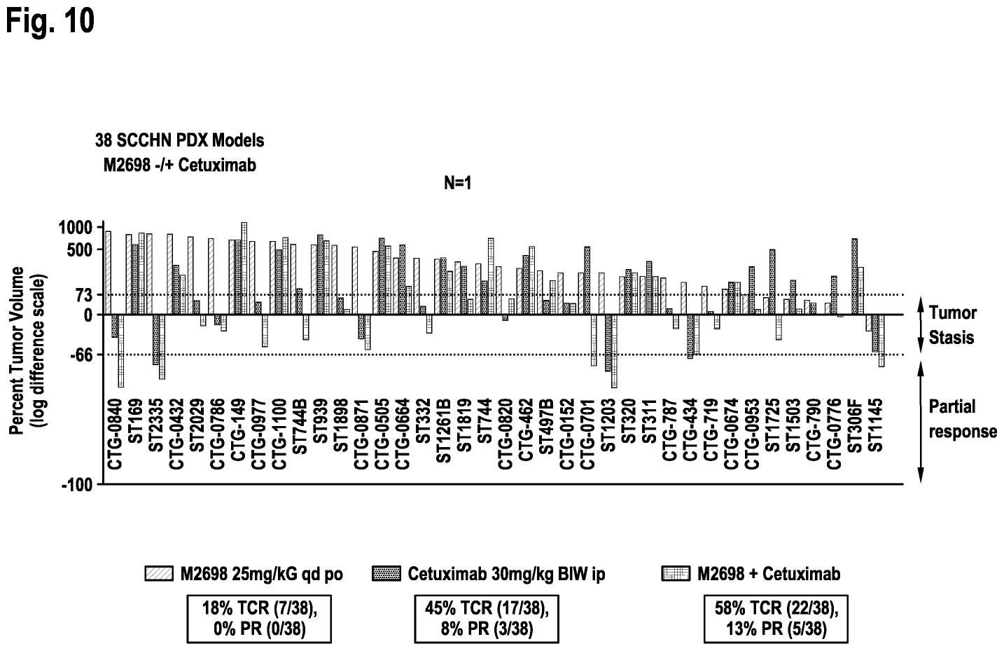

As the table above and FIG. 10 shows, for the combination, the TCR is 58% (22/38), which is an unexpected improvement of the TCR's of the single agents in this difficult to treat cancer type.

1: A compound mixture, comprising:
4-[(S)-2-Azetidin-1-yl-1-(4-chloro-3-trifluoromethyl-phenyl)-ethylamino]-quinazoline-8-carboxylic acid amide, or a physiologically acceptable salt thereof, and
an MEK inhibitor or a physiologically acceptable salt thereof.

2: The compound mixture according to claim 1, wherein the MEK inhibitor is pimasertib.

3: The compound mixture according to claim 1, further comprising an EGFR inhibitor.

4: A pharmaceutical composition, comprising:
the compound mixture according to claim 1, and
optionally, excipients and/or adjuvants.

5: A set (kit), comprising separate packs of:
(a) an effective amount of 4-[(S)-2-Azetidin-1-yl-1-(4-chloro-3-trifluoromethyl-phenyl)-ethylamino]-quinazoline-8-carboxylic acid amide or a physiologically acceptable salt thereof, and
(b) an effective amount of a MEK inhibitor, or a physiologically acceptable salt thereof.

6: The set (kit) according to claim 5, further comprising a separate pack of
(c) an effective amount of an EGFR inhibitor or a physiologically acceptable salt thereof.

7: The set (kit) according to claim 6, wherein the EGFR inhibitor is cetuximab.

8: A method for prophylaxis or treatment of cancer, the method comprising:
administering to a subject in need thereof, 4-[(S)-2-Azetidin-1-yl-1-(4-chloro-3-trifluoromethyl-phenyl)-ethylamino]-quinazoline-8-carboxylic acid amide, or a physiologically acceptable salt thereof, and a MEK inhibitor, or a physiologically acceptable salt thereof.

9: The method according to claim 8, wherein the MEK inhibitor is pimasertib.

10: The method according to claim 8, further comprising administering to the subject in EGFR inhibitor, or a physiologically acceptable salt thereof.

11: The method according to claim 10, wherein the EGFR inhibitor is cetuximab.

12: The method according to claim 8, wherein 4-[(S)-2-Azetidin-1-yl-1-(4-chloro-3-trifluoromethyl-phenyl)-ethylamino]-quinazoline-8-carboxylic acid amide, the MEK inhibitor and, optionally, an EGFR inhibitor, are administered simultaneously.

13: The method according to claim 8, wherein 4-[(S)-2-Azetidin-1-yl-1-(4-chloro-3-trifluoromethyl-phenyl)-ethylamino]-quinazoline-8-carboxylic acid amide, the MEK inhibitor and, optionally, an EGFR inhibitor, are administered sequentially.

14: A compound mixture, comprising:
4-[(S)-2-Azetidin-1-yl-1-(4-chloro-3-trifluoromethyl-phenyl)-ethylamino]-quinazoline-8-carboxylic acid amide, or a physiologically acceptable salt thereof, and
an EGFR inhibitor.

15: The compound mixture according to claim 14, wherein the EGFR inhibitor is cetuximab.

16: A set (kit), comprising separate packs of:
(a) an effective amount of 4-[(S)-2-Azetidin-1-yl-1-(4-chloro-3-trifluoromethyl-phenyl)-ethylamino]-quinazoline-8-carboxylic acid amide or a physiologically acceptable salt thereof, and
(b) an effective amount of an EGFR inhibitor.

17: The set (kit) according to claim 16, wherein the EGFR inhibitor is cetuximab.

18: A method for prophylaxis or treatment of cancer, the method comprising:
administering to a subject in need thereof, 4-[(S)-2-Azetidin-1-yl-1-(4-chloro-3-trifluoromethyl-phenyl)-ethylamino]-quinazoline-8-carboxylic acid amide, or a physiologically acceptable salt thereof, and an EGFR inhibitor.

19: The method according to claim 18, wherein the EGFR inhibitor is cetuximab.

20: The method according to claim 18, wherein 4-[(S)-2-Azetidin-1-yl-1-(4-chloro-3-trifluoromethyl-phenyl)-ethylamino]-quinazoline-8-carboxylic acid amide and the EGFR inhibitor are administered simultaneously.

21: The method according to claim 8, wherein 4-[(S)-2-Azetidin-1-yl-1-(4-chloro-3-trifluoromethyl-phenyl)-ethylamino]-quinazoline-8-carboxylic acid amide and the EGFR inhibitor are administered sequentially.

22: The method according to claim 8, wherein the cancer is colorectal cancer, breast cancer, cholangiocarcinoma, glioblastoma multiforme, squamous cell carcinoma of the head and neck, or non-small hug cancer.
